(19) United States
(12) Patent Application Publication
(10) Pub. No.: US 2024/0400996 A1
(43) Pub. Date: Dec. 5, 2024

(54) DENTAL PLURIPOTENT STEM CELLS

(71) Applicant: THE UNIVERSITY OF JORDAN, Amman (JO)

(72) Inventors: Abdalla AWIDI, Amman (JO); Dua'a ABUARQOUB, Amman (JO); Hanan JAFAR, Amman (JO)

(73) Assignee: THE UNIVERSITY OF JORDAN, Amman (JO)

(21) Appl. No.: 18/695,097

(22) PCT Filed: Sep. 23, 2021
(86) PCT No.: PCT/JO2021/050015

§ 371 Date: Mar. 25, 2024

(51) Int. Cl. C12N 5/0775; C12N 5/00

(52) U.S. Cl. CPC C12N 5/0664; C12N 5/0081; C12N 2502/115

(57) ABSTRACT

The present disclosure provides stem cells sorted from apical papilla tissue of impacted third molars and immature teeth of a subject, wherein the stem cells have pluripotent characteristics that can be differentiated into an ectoderm, a mesoderm, and endoderm germ layers, and wherein the stem cells are characterized by presence of NANOG, TRA-1-60, TRA-1-80, SSEA4, and OCT4 surface markers. The present disclosure further provides a method of isolating, sorting, and expanding such cells.

### TECHNICAL FIELD

The present disclosure relates to pluripotent stem cells, and more particularly to pluripotent stem cells obtained from the apical papilla tissue of impacted third molars (immature teeth of a patient).

### BACKGROUND

Stem cell therapy is a remarkable strategy in regenerative medicine and tissue engineering. The main types of human pluripotent stem cell (“PSC”) types are embryonic stem cells (“ESCs”) and induced pluripotent stem cells (“iPCSs”). Pluripotent stem cells have the capacity of self-renew and development into all three primary germ layers; endoderm, mesoderm and ectoderm, thus all adult cell types.

Human mesenchymal stem cells (“MSCs”) are multipotent cells that are isolated from adult tissues; bone marrow, adipose tissue, umbilical cord, dental pulp, peripheral blood, amniotic fluid and endometrium. These MSCs possess the potential to differentiate into a limited number of cell-lineages such as: osteoblasts, myocytes, chondrocytes and adipocytes as well as insulin-producing cells. To date, several types of human dental stem/progenitor cells have been isolated, identified and characterized. These cells are derived from neural crest derived tissue, and originated from ectodermal layer, therefore, they are called “ectomesenchyme”. These cells are classified into different groups according to the tissue from where these cells were isolated; Dental pulp stem cells (“DPSCs”), stem cells from exfoliated deciduous teeth (“SHEDs”), periodontal ligament stem cells (“PDLSCs”), dental follicle progenitor cells (“DFPCs”) and stem cells from apical papilla (“SCAPs”). These cells, like other types of isolated mesenchymal stem cells, have the capacity to differentiate into multiple lineages, including adipose, bone, endothelial and neural-like tissues.

Apical papilla tissue surrounds the apices of developing teeth near epithelial root sheath and are present in young impacted third molars (wisdom teeth). Third molar teeth are structurally not completely developed and present more immature tissue than dental pulp. Apical papilla contains higher density of mesenchymal stem cells than mature adult dental pulp tissue, thus a population presenting high proliferation and germination capacity.

Stem cell from apical papilla (“SCAP”) can differentiate into several functional tissue and embryonic layers. Specifically, SCAP possess markers of multipotent stem cells, properties of self-renewal potential, proliferation rate, migration, differentiation and low immunogenicity.

### SUMMARY

Based on these findings, the present disclosure suggests that the dental stem cells derived from the apical papilla tissue may have a unique population of cells with pluripotent properties morphological appearance and expression profile, and can differentiate into cells of all three germ layers; ectoderm, mesoderm and endoderm.

The present disclosure provides stem cells obtained from apical papilla tissue of impacted third molars and immature teeth of a subject, wherein the stem cells have pluripotent characteristics that can be differentiated into an ectoderm, a mesoderm, and endoderm germ layers, and wherein the stem cells are characterized by their morphological appearance (nucleus to cytoplasm ratio), in addition to the expression of pluripotency markers; NANOG, TRA-1-60, TRA-1-80, SSEA4, and OCT3/4 surface markers.

In aspects of the present disclosure, the subject may be a human.

Further aspects of the present disclosure provide a method of obtaining pluripotent stem cells, the method may include the steps of:
Obtaining extracted impacted third molars from a subject, followed by isolating the apical papilla tissue from the impacted third molars;
Culturing a fragmented apical papilla tissue in a plate comprising essential 8 flex media and alpha-modification of Eagle's Medium supplemented with glutamine penicillin, streptomycin, amphotericin B, and platelet lysate;
Incubating the cultured fragmented tissue in a humidified incubator in a tissue culture well plate coated with vitronectin;
Isolating stem cells from the obtained tissue followed by expanding the isolated stem cells;
Isolating a mixture of stem cells from apical papilla; and
Sorting a pure population of pluripotent stem cells from apical papilla.

In aspects of the present disclosure, sorting the isolated pluripotent stem cells from apical papilla may include the steps of:
Sorting the isolated pluripotent stem cells from apical papilla using a cell sorting device; and
Investigating the sorted pluripotent stem cells for a percentage of TRA-1-60 surface marker positive population;

Sorting the isolated pluripotent stem cells from apical papilla may further include culturing sorted pluripotent stem cell population in a plate coated with vitronectin and maintaining such population in essential 8 flex media.

### BRIEF DESCRIPTION OF THE DRAWINGS

The disclosure will now be described with reference to the accompanying drawings, which illustrate embodiments of the present disclosure without however limiting the scope of protection thereto, and in which:

FIG. 1A is a flowchart of a method of obtaining pluripotent stem cells from apical papilla tissue, the method being configured in accordance with embodiments of the present disclosure.

FIG. 1B illustrates a flowchart of a method for sorting isolated pluripotent stem cells, the method being configured in accordance with embodiments of the present disclosure.

FIG. 2A is a presentation of morphological appearance of colonies of pluripotent stem cells isolated from apical papilla (“PSCAP”) at a magnification factor of about 10.

FIG. 2B is a representation of morphological appearance of colonies PSCAP at a magnification factor of about 20.

FIG. 2C is an image of PSCAP under transmission electron microscopy exhibiting large nuclei and small cytoplasmic volume.

FIG. 3 is a flow cytometric analysis of (PSCAP) (solid line peaks), for pluripotency markers: Nanog; TRA1-60; SSEA4; OCT3/4 and TRA1-80, in comparison with their isotype controls (dashed line peaks).

FIG. 4A is an image taken by fluorescent microscopy by using FITC filter for Alkaline phosphatase (ALP) non-stained derived PSCAP.

FIG. 4B is an image taken by fluorescent microscopy by using FITC filter for ALP stained derived PSCAP.

FIG. 5 is an RT-PCR analysis of pluripotency genes: OCT3/4; SOX2; Nanog, and PPIA (housekeeping gene) of PSCAP.

FIG. 6 is an image taken by using Confocal microscopy of immunofluorescence-stained pluripotency markers for: SSEA4; OCT3/4; SOX1 and TRA1-60 of PSCAP

FIG. 7A is an image taken using inverted microscopy of Alizarin red stained calcium deposits formed PSCAP after 18 days of osteogenic differentiation for induced cells cultured in osteogenic media as an example of mesodermal differentiation.

FIG. 7B is an image taken using inverted microscopy of Alizarin red stained calcium deposits formed PSCAP after 18 days of osteogenic differentiation for control (uninduced cells) cultured in cell culture media.

FIG. 8 is a chart illustrating the identification of the expression of the osteogenic marker genes; Osteonectin, cbfa-1, Collagen type 1, and Osterix.

FIG. 9 presents images taken by fluorescent Microscopy at a magnification factor of about 25 of neurogenic markers in PSCAP after 7 days of neurogenic induction. A-1) nuclear staining with DAPI; A-2) Nestin; A-3) Sox1; A-4) merge of DAPI, Nestin and SOX1; B-1) nuclear staining with DAPI; B-2) PAX; B-3) SOx2; B-4) merge of PAX, SOX1 and DAPI; C-1) nuclear staining with DAPI; C-2) β TubulinIII (TUJ1); C-3) merge of β TubulinIII (TUJ1) with DAPI.

FIG. 10 is a flow cytometric analysis of PSCAP after definitive endoderm induction in comparison to the uninduced cells A) SOX17-PE; B) FOXA2-AF647. PSCAP differentiated cells (solid line peaks), undifferentiated cells (dashed line peaks).

FIG. 11 is an image of islet-like clusters of PSCAP after 14 days of beta cell differentiation, stained with Diphenylthiocarbazone (“DTZ”). A) induced cells; B) Control-(uninduced cells).

FIG. 12 is a presentation of images taken by fluorescent microscopy of immunofluorescence-stained Pancreatic Beta cell markers: Insulin; PDX-1 and somatostatin in pluripotent stem cells isolated from apical papilla (PSCAP) 1 after 14 days of beta cell differentiation.

### DETAILED DESCRIPTION

In view of the foregoing, it is an objective of the present disclosure to provide PSCAP, and a method for isolating young developed pluripotent stem cells. To this end, apical papilla-mediated stem cell regeneration offers opportunities to regenerate all three germ layers; ectoderm, mesoderm and endoderm.

In the present disclosure, a novel population PSCAP that is capable of differentiating into osteogenic, neurogenic and β-cell differentiation cells have been successfully isolated. The current invention characterized and sorted stem cells by the expression of pluripotency markers.

In embodiments of the present disclosure, characterization of pluripotent stems cells may be applied to surface, intracellular and extracellular molecules, wherein such characterization of PSCAPs may be applied to markers of embryonic stem cells.

Similar to embryonic stem cells, PSCAPs express one or more cell surface markers as SSEA-4, TRA-1-60, TRA-1-80 and may express TRA-1-81, SSEA-1, OCT4, NANOG and other cell surface pluripotency markers.

Similar to embryonic stem cells, PSCAPs may express one or more signal pathway-related intracellular markers including but not limited to LIF-STAT3, BMP-SMAD, TGF-β/Activin/Nodal, IGF-IR, FGFR and Wnt-β-catenin signal pathways.

Similar to embryonic stem cells, PSCAPs may express other small molecules including but not limited to lectins, short peptides, peptides and other carbohydrate-binding proteins.

Similar to embryonic stem cells, PSCAPs express enzymatic marker alkaline phosphatase.

In yet another embodiment, the dental tissue, apical papilla tissue is isolated in a culture medium, that is used to culture pluripotent stem cells.

Also provided herein are culture media compositions and kits useful for inducing germ layer differentiation and screening assays for agents that can modulate the differentiation of the cells or for primary or secondary screens of the cells generated by the methods described herein.

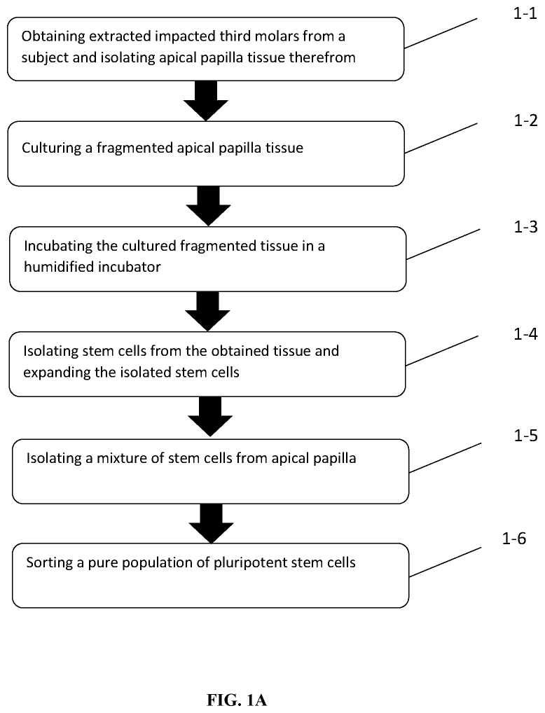

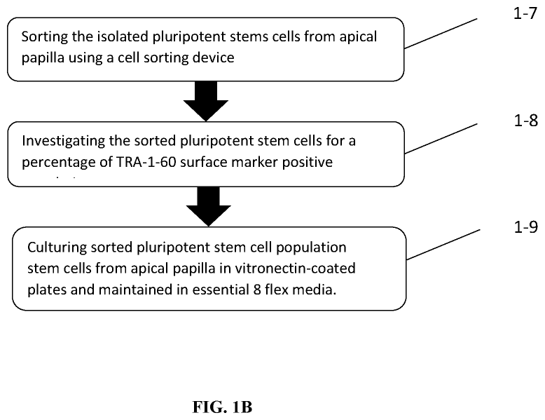

Reference is now being made to FIG. 1A, which illustrates a method of obtaining the pluripotent stem cells from apical papilla in accordance with embodiments of the present disclosure, the method may include the steps of obtaining extracted impacted third molars from a subject, followed by isolating the apical papilla tissue from the impacted third molars (process block 1-1); followed by culturing a fragmented apical papilla tissue in a plate including essential 8 flex media and alpha-modification of Eagle's Medium supplemented with glutamine penicillin, streptomycin, amphotericin B, and platelet lysate (process block 1-2); incubating the cultured fragmented tissue in a humidified incubator in a tissue culture well plate coated with vitronectin (process block 1-3); isolating stem cells from the obtained tissue followed by expanding the isolated stem cells (process block 1-4); isolating a mixture of stem cells from apical papilla (process block 1-5); and sorting a pure population of pluripotent stem cells from apical papilla (process block 1-6).

In some embodiments, the amount of essential 8 flex media is about 50% by volume.

In embodiments of the present disclosure, the amount of alpha-modification of Eagle's Medium is about 50% by volume.

In some embodiments, the concentration of glutamine is about 2 mM.

In some embodiments, the concentration of penicillin is 100 units/mL.

In some embodiments, the concentration of streptomycin is 100 mg/mL.

In some embodiments, the concentration of amphotericin B is 0.25 mg/mL.

Referring now to FIG. 1B, the sorting of the isolated pluripotent stem cells from apical papilla may include the steps of sorting the isolated pluripotent stem cells from apical papilla using a cell sorting device (process block 1-7); and investigating the sorted pluripotent stem cells for a percentage of TRA-1-60 surface marker positive population (process block 1-8).

In some embodiments, sorting the isolated pluripotent stem cells from apical papilla may further include culturing sorted pluripotent stem cell population in a plate coated with vitronectin and maintaining such population in essential 8 flex media (process block 1-9).

In embodiments of the present disclosure, the low temperature at which the cell pellet is incubated may be about 4° C.

In embodiments of the present disclosure, centrifugation is performed for about 5 minutes at about 200×g.

In some embodiments of the present disclosure, the volume of phosphate buffer saline is about 500 μL.

Accordingly, the present disclosure provides a method of isolating and expanding pluripotent stem cell of apical papilla tissue, including the steps of:
(a) in vitro isolation of stem cells of apical papilla by the methods described herein; and
(b) subjecting isolated stem cells to expansion and differentiation.

In one embodiment, the differentiation and screening of germ layer cells may include the steps of
(a) preparing a culture of ectodermal, mesodermal and/or endodermal germ layer cells by the methods described herein;
(b) subjecting the differentiated germ layer cells to analysis.

In another embodiment, there is provided a method of inducing and screening pancreatic endoderm progenitor cells comprising:
a) preparing a culture of pancreatic cells by the methods described herein;
b) subjecting the pancreatic cells to analysis.

The isolation medium can be any cell culture medium. In one embodiment, the isolation medium is a pluripotent stem cell maintenance medium. In another embodiment, the isolation medium comprises a mixture of a-MEM and feeder-free basal medium, optionally, further comprises platelet lysate, antibiotics, vitamins, salts, trace elements, lipids, proteins, amino acids or mixtures thereof. In another embodiment, the medium is Essential 8 Flex™, E8 or an essential feeder-free medium.

The expansion medium can be any cell culture medium. In one embodiment, the expansion medium is a pluripotent stem cell culture medium. In another embodiment, the expansion medium is a xeno-free and feeder-free basal medium. In another embodiment, the expansion medium comprises a feeder-free medium, optionally, further comprises antibiotics, vitamins, salts, trace elements, lipids, proteins, amino acids or mixtures thereof. In another embodiment, the medium is Essential 8 Flex™, E8 or an essential feeder-free medium.

In an embodiment, the culture plates for sorted pluripotent stem cells culture were Vitronectin coated surfaces.

Mainly, culture plates were coated with Vitronectin, optionally, a glycoprotein, blood glycoprotein, a molecule that binds to glycosaminoglycans, collagen, plasminogen or urokinase-receptor.

In an embodiment, the culture medium used for osteogenic differentiation of the human pluripotent stem cells to definitive mesoderm is an osteogenic medium (Gibco, USA), whereby the protocol is followed according to manufacturer's instructions.

In an embodiment, the culture medium used for osteogenic differentiation of the human pluripotent stem cells comprises a basal medium, glucose, glutamine, buffers which may comprise sodium phosphate, sodium bicarbonate, sodium pyruvate and osteogenesis supplements.

In an embodiment, the culture medium used for neurogenic differentiation of the human pluripotent stem cells to definitive ectoderm is a neural induction medium (Gibco, USA), whereby the protocol is followed according to manufacturer's instructions.

In an embodiment, the culture medium used for neural induction differentiation of the human pluripotent stem cells comprises a basal medium, glucose, glutamine, buffers which may comprise sodium phosphate, sodium bicarbonate, sodium pyruvate and neurogenesis supplements.

In an embodiment, the culture medium used to differentiate the human pluripotent stem cells to definitive endoderm is a PSC definitive endoderm induction medium (Thermofisher scientific, USA), whereby the protocol is followed according to manufacturer's instructions.

In an embodiment, the culture medium used for endoderm induction differentiation of the human pluripotent stem cells comprises a basal medium, glucose, glutamine, buffers which may comprise sodium phosphate, sodium bicarbonate, sodium pyruvate and endoderm supplements.

In an embodiment, the culture medium used to differentiate the endoderm layer to specific pancreatic definitive endoderm cells is Advanced DMEM-F12 (Gibco, USA).

In an embodiment, the culture medium used for pancreatic endoderm induction differentiation of the endoderm layer comprises a basal medium, bovine serum albumin, insulin-transferrin-sodium, Taurine, serum, glucagon-like peptide, nicotinamide, buffers which may comprise sodium phosphate, sodium bicarbonate, sodium pyruvate and endoderm supplements.

The disclosure further relates to a cell culture medium useful for culturing cells of the mesenchymal cell lineage and/or enriching cells of the mesenchymal cell lineage and/or expansion of the mesenchymal cell into lineages and/or isolation of mesenchymal cells.

In one embodiment, the method further comprises obtaining a population of cells enriched for cells of the mesenchymal cell lineage.

The disclosure further relates to the cells from apical papilla and/or embryonic cells that may comprise cells of the mesenchymal cell lineage and non-mesenchymal cells.

In another embodiment, the tissue sample comprises dental tissue, compact teeth, dental root tissue, or any oral tissue where pluripotent stem cell reside.

In one embodiment, progenitor cell lineages possess pluripotent stem cell characteristics, such as the expression of pluripotency markers.

Accordingly, pluripotent stem cells can be sorted via phenotypic characterization, wherein, morphological appearance, activity of surface markers, expression of surface markers. Markers can be labelled with fluorophores or stains. Accordingly, precise cell identification techniques include flow cytometry sorting, immunofluorescence, immunohistochemistry, or visualization via transmission electron microscopy, morphological microscopy

Definitive markers can be determined molecularly, enzymatically and/or morphologically.

In yet another embodiment, the pluripotent cells are further differentiated into progenitor cells.

In yet another embodiment, progenitor cells differentiate into mesoderm, ectoderm and endoderm.

Mesoderm cells compose osteogenic cells, accordingly, osteogenic deposits, specifically calcium deposits are formed. In order to detect the mineralization pattern of the osteogenic cells, calcium deposits can be stained and observed under the microscope.

Osteogenic cells are fixed and stained for the calcium deposits; the stain can be a calcium binding stain. The stain is alizarin red stain.

Determination of osteogenic differentiation efficiency can be by detection of osteogenic markers. Osteogenic markers can be one or more of Osteonectin, cbfa-1, Collagen type 1, Osterix, OPN, OCN and ALP.

Ectoderm cells compose neurogenic cells; accordingly, neurogenic cells are neurospheres.

Neurogenic cells can be identified by the detection of neurogenic antigens, thus by antibody binding for immunohistochemistry and/or immunofluorescence, accordingly visualized under microscope.

Determination of neurogenic differentiation can be by detection of neurogenic markers. Neurogenic markers can be one or more of B-Tubulin, Nestin, SOX1, SOX2 and PAX6.

Endodermal cells are further differentiated to form pancreatic cells, respiratory cells, intestinal cells or liver cells.

Determination of pancreatic beta-cells can be by detection of definitive endoderm markers.

In yet another embodiment, the pancreatic cells may express one or more definitive endoderm markers, can be one or more of FoxA12, SOX17, PDX-1, and somatostatin. Accordingly, sorted by flow cytometry.

Pancreatic beta-cells compose pancreatic islets. Determination of the formation and maturation of islets like clusters can be accordingly, through Diphenylthiocarbazone staining and detection of maturation markers PDX-1 and somatostatin through immunofluorescence.

The disclosure is now further illustrated on the basis of Examples and a detailed description from which further features and advantages may be taken. It is to be noted that the following explanations are presented for the purpose of illustrating and description only; they are not intended to be exhaustive or to limit the disclosure to the precise form disclosed.

### Example 1

Isolation of Dental Stem Cells and Pluripotent Stem Cells from Apical Papilla (“PSCAP”)

To isolate stem cells of the apical papilla, submerged teeth were subjected to stem cell isolation within 24 hours of collecting the samples of volunteers; and third molars were incubated with phosphate buffer saline PBS (Gibco, USA) supplemented with 10% P/S and Amphotericin B at room temperature for 15 minutes under aseptic conditions. Teeth were washed (three times) with PBS supplemented with 1% P/S and apical papilla tissue was cut away from the immature roots by using sterile scalpel and blade. Extracted tissue was then minced into small pieces, and small fragments were maintained in two different types of culture medium; a maintenance medium for pluripotent stem cells (PSCs) and, another maintenance medium for mesenchymal stem cells (MSCs). Cultured cells were incubated in a humidified incubator (Memmert, Germany) at 37° C. in 5% CO₂.

Referring back to FIG. 1B, after that, to isolate pluripotent stem cells from the apical papilla, the fragmented tissue was cultured in a tissue culture 6 well plate coated with vitronectin (Gibco, USA) that contained 50% Essential 8 Flex media (Gibco, USA), in addition to 50% of alpha MEM media (Gibco, USA), supplemented with 2 mM glutamine (Glutamax, Invitrogen, USA), 100 U/ml penicillin, 100 mg/ml streptomycin, 0.25 mg/ml Amphotericin B and 5% platelet lysate (PL) (process block 1-5). The cells were incubated in a humidified incubator (Memmert, Germany) at 37° C. and 5% CO₂ and medium was exchanged day after day (process block 1-6). Once cells reached 80%-90% confluence, cells were subcultured with Accutase (Gibco, USA) and prepared for sorting (process block 1-7).

### Example 2

Sorting of Pluripotent Stem Cells from Apical Papilla

In order to isolate pure culture of pluripotent stem cells, the derived stem cells from apical papilla (PSCAP) were investigated for the percentage of TRA1-60 positive population (process block 1-9), by sorting using FACS JAZZ cell sorter (BD Biosciences, USA) (process block 1-8). Sorted populations were divided into two divisions based on the expression of TRA1-60: TRA1-60+ve and TRA1-60−ve. Sorted cells were cultured at 37° C. and 5% CO₂ in a tissue culture 24 well plate coated with vitronectin, and maintained in 100% Essential 8 flex media only. Further, PSCAP were seeded at a density of 1×10³ cells/cm²

### Example 4

### Phenotypic Characterization

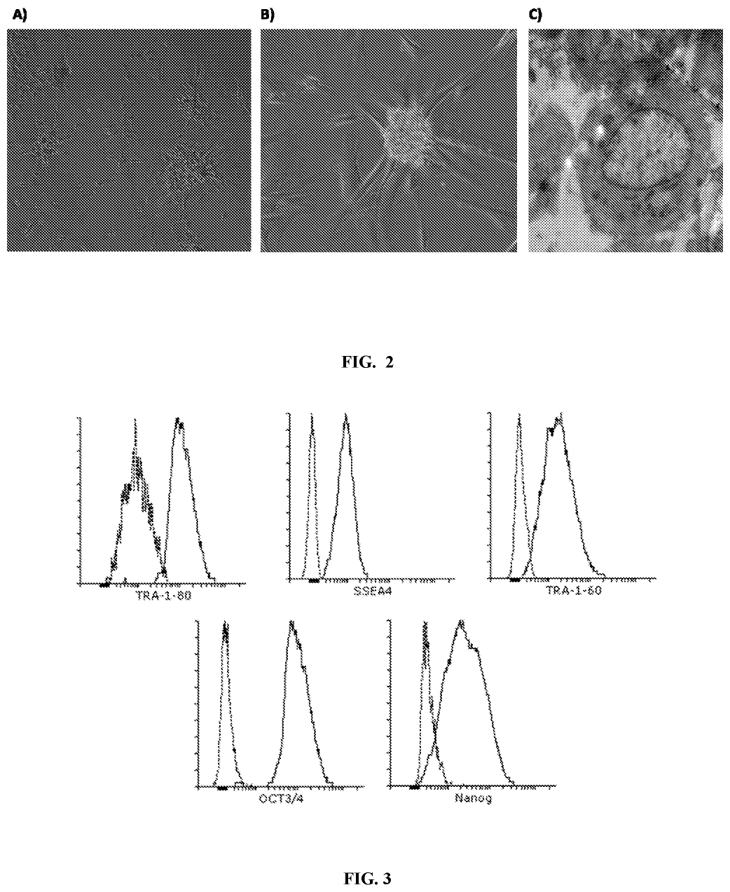

In order to determine the morphological appearance, time of growth and time of reaching confluence of the isolated cells, sorted PSCAP were examined under an inverted microscope (Axiovert, Zeiss, Germany). Furthermore, to investigate the differences in the measurements of the derived cells: diameter, cytoplasmic volume and nucleus size, sorted PSCAP were screened by using transmission electron microscopy (TEM). Colonies of pluripotent stem cells were observed under microscopy as pointed in FIGS. 2A, 2B. The cytoplasmic volume was confirmed small and the nucleus size was indicated larger as shown in FIG. 2C.

### Example 5

### Characterization of the Pluripotency Status Based on the Expressed Markers

To identify the expression of the preferred pluripotency genes; OCT3/4, SOX2 and Nanog, PCR was performed for the sorted PSCAP. RNA was extracted from the sorted PSCAP, reverse transcribed into cDNA and preferred genes amplified. The PCR product was run on a 2% agarose gel (2 g agarose in 100 ml TBE buffer) and presence of bands were indicative of pluripotent marker expression, as shown in FIG. 5.

### Example 6

### Alkaline Phosphatase Staining (“ALP”) of Pluripotent Cells

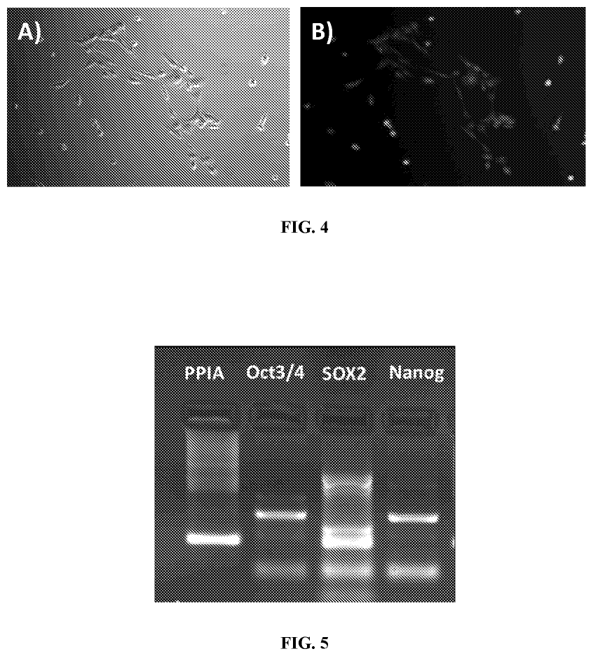

To determine the expression of ALP, which is highly elevated in the pluripotent stem cells, sorted PSCAP, were cultured in plates (IBID, USA), stained with the live ALP staining (Thermofisher scientific, USA) based on the manufacturer instructions. Cells were observed under Zeiss Axio Observer.Z1 Fluorescence Microscope (Zeiss, Germany) by using standard FITC filter. Colonies of pluripotent cells (FIG. 4A showed marked presence of ALP within the cells, a phenotypic marker, shown in FIG. 4B.

### Example 7

### Detection of Pluripotency Markers

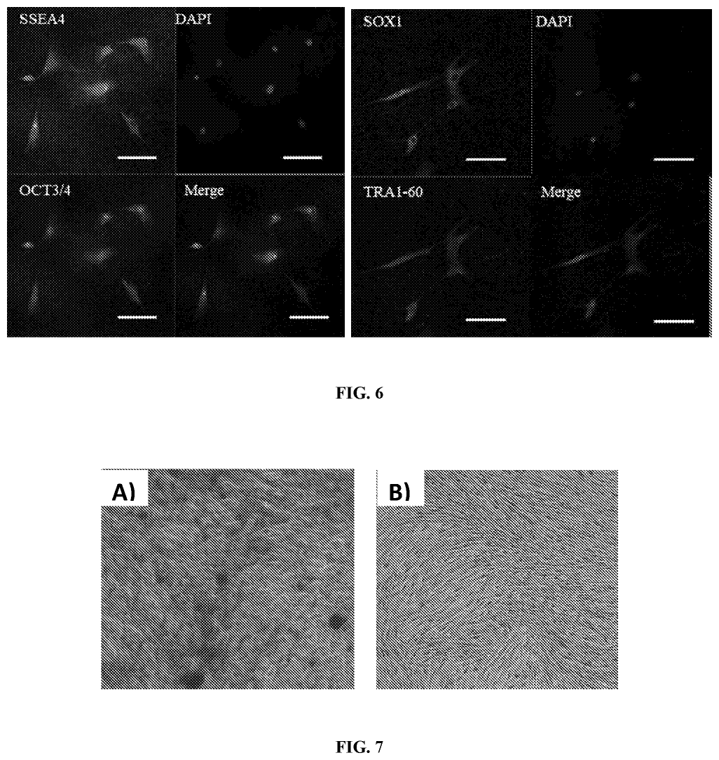

To identify the expression of the preferred pluripotency markers: TRA1-60, SSEA4, TRA1-80, Nanog SOX1 and OCT3/4 in the sorted PSCAP, the expression profile of pluripotency markers for all cell types was analyzed by flow cytometric analysis by Flourescien activated cell sorter FACS Canto II, FACS Diva software version 8 (BD, USA). Results of flow cytometric analysis, presented in FIG. 3, showed marked expression of pluripotency markers in comparison with their isotype controls. Furthermore, cells were treated according to PSC 4-Marker Immunocytochemistry Kit PSC (OCT4, SSEA4, SOX2, and TRA-1-60), stained cells were observed under Zeiss Axio Observer.Z1 Fluorescence Microscope (Zeiss, Germany). In reference to FIG. 6 presenting immunofluorescence staining of extracted pluripotent stem cells of the apical papilla, cells expressed pluripotency markers SSEA4, OCT3/4, SOX1 and TRA1-60 in its cellular makeup.

### Example 8

### Mesoderm Induction (Osteogenic Differentiation) and Validation

In order to induce osteogenic differentiation, sorted pluripotent stem cells (PSCAP) at P3 were cultured in 6 well plates coated with Vitronectin (Gibco, USA) and maintained in Essential 8 Flex medium (Gibco, USA). Once 50% confluence was reached, media was replaced with osteogenic medium (Gibco, USA) and cells were incubated for 18 days. Media was exchanged twice a week and cells cultured in Essential 8 flex medium were used as negative control.

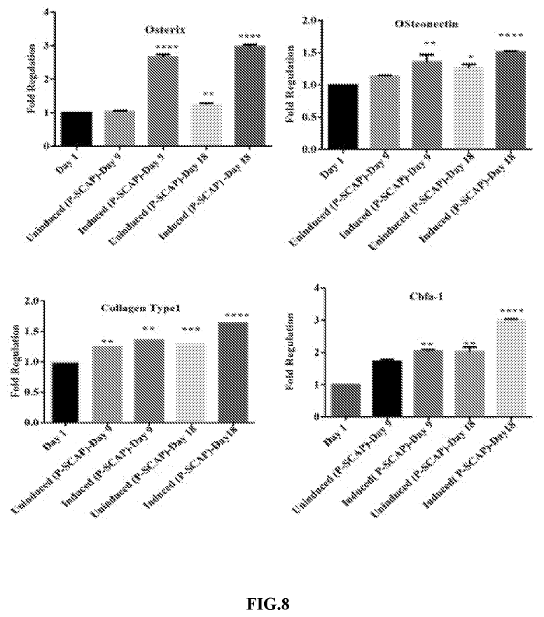

In order to confirm differentiation into osteogenic cells, and detect the mineralization pattern of the differentiated cells; cells were subjected to calcium deposits staining by Alizarin Red Stain (ARS), visualized and observed under the inverted microscope (Axiovert, Zeiss, Germany). Accordingly, as shown in FIG. 7, after 18 days of osteogenic differentiation of pluripotent stem cell from the apical papilla, cells have formed calcium deposits which confirm its differentiation into mesoderm germ layers. In addition, cells were also subjected to PCR reactions to identify the expression of the preferred osteogenic marker genes; Osteonectin, cbfa-1, Collagen type 1, Osterix, as shown in FIG. 8. Furthermore, to validate osteogenic marker expression, cells were subject to ELISA to evaluate efficiency of osteogenic differentiation by preferred osteogenic markers (OPN, OCN and ALP). As shown in Table 1 below, induced pluripotent stem cells from the apical papilla expressed marked increase in OPN, OCN and ALP throughout the induction duration (18 days) in comparison the uninduced pluripotent stem cells.

TABLE 1

| | OPN | OCN | ALP |
|---|---|---|---|
| Sample Name | (ng/ml) | (ng/ml) | (nmol/well) |
| Induced-(PSCAP) Day 1 | 0.713 | 0.807 | 0.257 |
| Induced-(PSCAP) Day 9 | 1.443 | 1.537 | 0.261 |
| Induced-(PSCAP) Day 18 | 1.494 | 1.793 | 0.428 |
| Uninduced-(PSCAP) Day 1 | 0.713 | 0.807 | 0.257 |
| Uninduced-(PSCAP) Day 9 | 1.087 | 1.180 | 0.194 |
| Uninduced-(PSCAP) Day 18 | 1.101 | 1.370 | 0.154 |

### Example 9

### Ectoderm Induction (Neurogenic Differentiation) and Validation

In order to induce neurogenic differentiation, sorted pluripotent stem cells (PSCAP) at P3 were cultured in 6 well plates coated with Vitronectin (Gibco, USA). Once 70% confluence was reached, cells were detached by using Accutase (Gibco, USA), centrifuged at 200×g for 4 min and the supernatant was discarded. Pellet was resuspended in PSC neural induction medium (Gibco, USA) and transferred into ultra-low attachment 6 well plate (Corning, USA) at 0.5×10⁶ cells/well. Cells cultured in the essential 8 flex medium (Gibco, USA) were used as a negative control and Human gliobastoma cell line U-87 MG (ATCC® HTB-14™) was used as a positive control. Cells were maintained in neurogenic medium for 8 days, and medium was exchanged every other day.

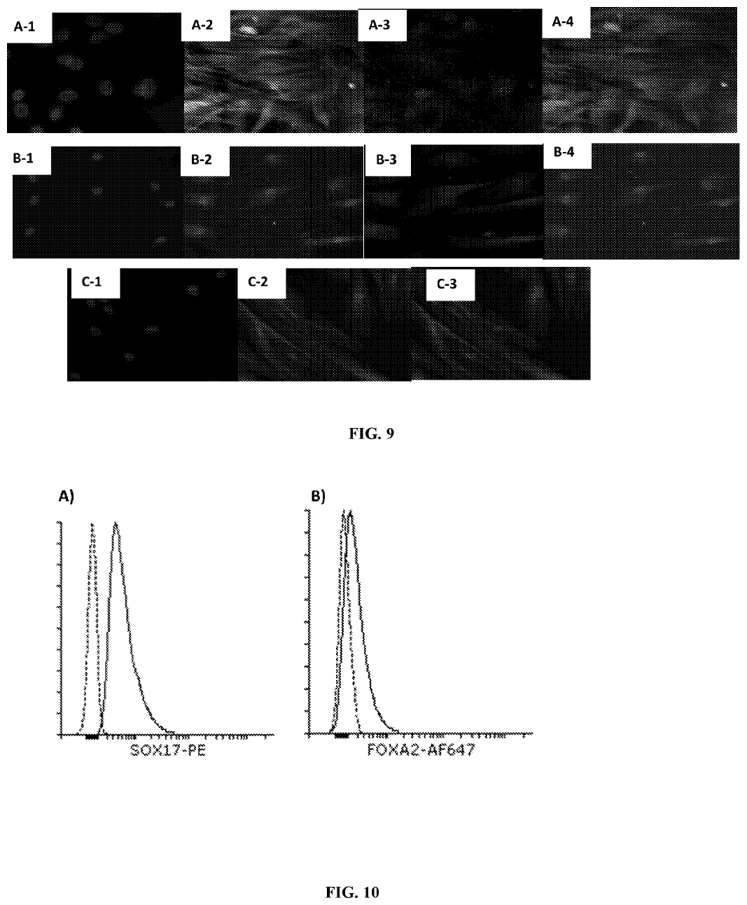

In order to confirm differentiation into neurogenic cells; cells were subject to immunofluorescence determination, to identify the expression of the preferred neurogenic marker genes; B-Tubulin, Nestin, SOX1, SOX2 and PAX6. As shown in FIG. 9, staining with corresponding fluorophores of neurogenic markers showed expression of β-Tubulin, Nestin, SOX1, SOX2 and PAX6 under fluorescent microscopy, thus, confirming differentiation of ectoderm germ layer.

### Example 10

### Endoderm Differentiation, Induction of Pancreatic Beta Cells and Validation

In order to produce the definitive endoderm layer as a starting point of Beta cell differentiation, sorted P-SCAP cells were cultured in 6 well plates coated with Vitronectin (Thermo Fisher Scientific, USA) and maintained with Essential 8 flex media (Gibco, USA). Once 50-60% confluence was reached cells were induced by replacing the Essential 8 flex medium by PSC Definitive Endoderm (DE) Induction kit (Thermofisher scientific, USA). At day 1, PSC Definitive Endoderm (DE) Induction media A was added to the PSCAP, and at day 2, PSC Definitive Endoderm (DE) Induction media A was replaced with PSC Definitive Endoderm (DE) Induction media B.

In order to determine the effectiveness of the differentiation into definitive endoderm, cells were analyzed by flow cytometric analysis to identify the expression of the preferred definitive endoderm markers; FOXA2 and SOX17. According to the results interpreted in FIG. 11, induced pancreatic cells showed marked expression of SOX17 and FOXA2 which indicates successful induction of the pancreatic endoderm germ layer.

In order to induce the differentiation of Endoderm cells into a specific line of endoderm (Pancreatic differentiation), endoderm germ layer cells were induced by adding Advanced DMEM-F12 (Gibco, USA), supplemented with 1% BSA (Biowest, USA), 1% ITS (Sigma, USA), 0.3 mM Taurine (Sigma, USA) and without serum for 2 days. At day 5, differentiating functional pancreatic cells were induced by Advanced DMEM-F12 (Thermofisher scientific, USA), supplemented with 1.5% BSA (Biowest, USA), 1.5% ITS (Sigma, USA), 3 mM Taurine (Sigma, USA), 100 nM Glucagon-like peptide (Sigma, USA) and 1 mM nicotinamide (Sigma, USA) without serum for 10 days. Medium was exchanged every 2 days.

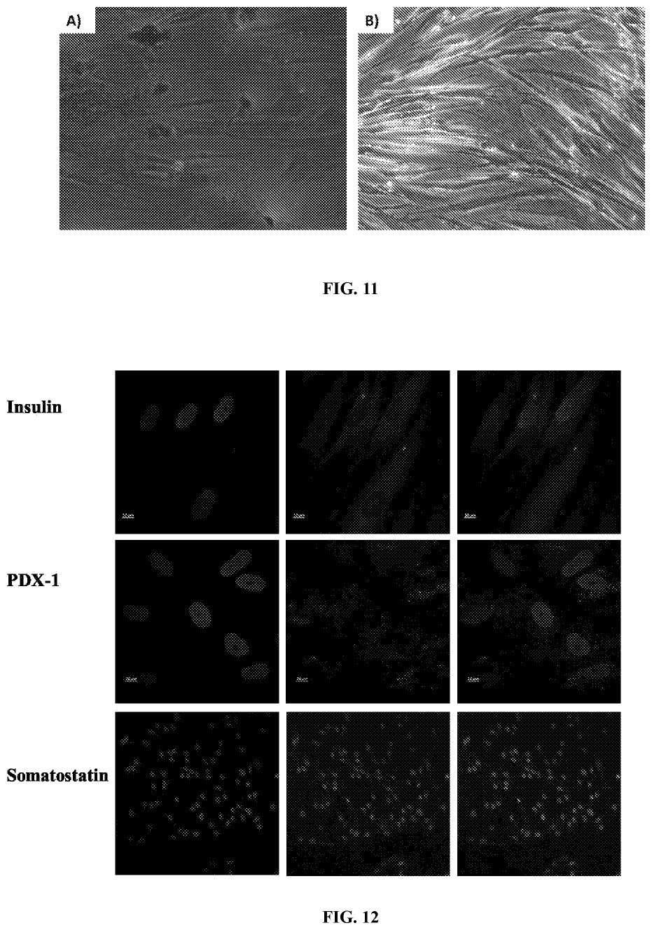

In order to determine the formation and maturity of pancreatic islet-like clusters, cells were subject to Diphenylthiocarbazone staining (DTZ), and to immunofluorescence staining for preferred pancreatic cell maturation markers; Insulin, PDX-1 and somatostatin. After 14 days from exposure to pancreatic differentiation medium, cells stained with DTZ showed marked formation of islet-like clusters (shown in FIG. 10), thus, showed definitive marker for the formation of pancreatic endoderm germ layer. Cells were capable of secreting insulin, as shown in FIG. 12, after staining with corresponding stains. Furthermore, pancreatic cell maturation markers; PDX-1 and somatostatin were evident in cells as observed under immunofluorescence staining (FIG. 12).

To determine the maturity of the differentiated cells into islets like clusters, at day 14 of differentiation, cells were fixed by 4% paraformaldehyde for 20 minutes. Then, washed with PBS and stained with Diphenylthiocarbazone (DTZ, Sigma, USA) for 20 minutes. Later, fixed cells were observed under the inverted microscope (Axiovert, Ziess, Germany).

What is claimed is:

1. Stem cells obtained from apical papilla tissue of impacted third molars of a subject, wherein the stem cells have pluripotent characteristics that can be differentiated into an ectoderm, a mesoderm, and endoderm germ layers, and wherein the stem cells are characterized by presence of NANOG, TRA-1-60, TRA-1-80, SSEA4, and OCT4 surface markers.

2. The stem cells of claim 1, wherein the subject is a human.

3. A method of obtaining the stem cells of claim 1 comprising the steps of:
obtaining extracted third molars from a subject, followed by isolating the apical papilla tissue from the impacted third molars;
culturing a fragmented apical papilla tissue in a plate including essential 8 flex media and alpha-modification of Eagle's Medium supplemented with glutamine penicillin, streptomycin, amphotericin B, and platelet lysate;
incubating the cultured fragmented tissue in a humidified incubator in a tissue culture well plate coated with vitronectin;
isolating stem cells from the obtained tissue followed by expanding the isolated stem cells;
isolating a mixture of stem cells from apical papilla; and
sorting a pure population of pluripotent stem cells from apical papilla.

4. The method of claim 3, wherein sorting the isolated pluripotent stem cells from apical papilla comprises the steps of:
Sorting the isolated pluripotent stem cells from apical papilla using a cell sorting device; and
Investigating the sorted pluripotent stem cells for a percentage of TRA-1-60 surface marker positive population.

5. The method of claim 4, further comprising culturing sorted pluripotent stem cell population in a plate coated with vitronectin and maintaining such population in essential 8 flex media.
